(19) United States
(12) Patent Application Publication
(10) Pub. No.: US 2008/0038267 A1
(43) Pub. Date: Feb. 14, 2008

(54) Treatment Of Cancer

(75) Inventors: James Peter Burnie, Manchester (GB); Ruth Christine Matthews, Manchester (GB); Tracey Carter, Manchester (GB)

Correspondence Address: FISH & RICHARDSON PC, P.O. BOX 1022, MINNEAPOLIS, MN, 55440-1022, US

(73) Assignee: NEUTEC PHARMA LIMITED, Surrey (GB)

(21) Appl. No.: 11/630,936

(22) PCT Filed: Jun. 30, 2005
(86) PCT No.: PCT/GB05/02545

§ 371 Date: Jul. 25, 2007

Related U.S. Application Data
(60) Provisional application No. 60/614,423, filed on Sep. 30, 2004.
(60) Provisional application No. 60/654,458, filed on Feb. 22, 2005.

(30) Foreign Application Priority Data
Jul. 2, 2004 (GB) 0414885.4
Sep. 20, 2004 (GB) 0420845.0
Feb. 21, 2005 (GB) 0503566.2

(51) Int. Cl. A61K 39/00; A61P 35/00

(52) U.S. Cl. 424/139.1

(57) ABSTRACT

The present invention relates to a novel medicaments and preparations comprising effective anti-cancer agents together with an anti-Hsp90 antibody which together provide an enhanced efficacy in the treatment of cancer, and leukaemia.

The present invention relates to novel medicaments and preparations comprising effective pharmaceutical agents together with an anti-Hsp 90 antibody which together provide an enhanced efficacy in the treatment of cancers, including colorectal cancer. Other aspects of the invention are concerned with the treatment of leukaemias.

Cancer Therapy and Treatment of Leukaemia

A first aspect of the present invention relates to novel medicaments and preparations comprising effective anti-cancer agents together with an anti-Hsp90 antibody which together provide an enhanced efficacy in the treatment of cancer.

Members of the heat shock proteins (Hsp) family of proteins have emerged in recent years as having an important role in oncogenesis and cell death. Indeed, heat shock proteins have been identified as being potential targets for cancer therapy for many years (Whitesell L et al., PNAS USA, 1994 Aug. 30, 91(18): 8324-8; PMID: 8078881), and members of the ansamycin family (formerly referred to as tyrosine kinase inhibitors) have been suggested as useful in effecting cancer therapy (Neckers L et al., Invest New Drugs, 1999, 17(4): 361-73; PMID: 10759403; Schulte T W et al., Cancer Chemother Pharmacol., 1998, 42(4): 273-9; PMID: 9744771).

One heat shock protein, Hsp90, has been implicated as involved in carcinoma of the breast, prostate, melanoma, leukaemias and lymphomas, colon and lung (Banerji U et al., Curr Cancer Drug Targets, 2003 October; 3(5): 385-90; PMID: 14529390), as well as thyroid carcinomas. The role of Hsp90 is to ensure the correct folding of “client proteins” which are involved in a wide variety of cellular processes, for example signal transduction. Hsp90 client proteins include transcription factors such as mutant p53 and hypoxia-inducible factor 1α, and soluble kinases including v-Src, Akt, Raf-1, and Bcr-Abl. Hsp90 is constitutively expressed at 2- to 10-fold higher levels in tumour cells than in normal cells, suggesting that it may be important for the growth/survival of tumour cells (Schwartz, J., et al., 2003, Semin. Hematol. 40:p 87-96). Since the binding of client proteins to Hsp90 can regulate their conformation, stability and fate in the cell, Hsp90 can have a major impact on the pathways that regulate cellular outcome, including cell growth, division, differentiation, movement and death (Workman, P., Cancer Lett. 2004 Apr. 8; 206(2):149-57; PMID: 15013520). The wide reaching role for Hsp90 in cellular processes means the protein is currently viewed as a possible target for the development of therapeutic drugs. Hsp 90 inhibitors, by specifically interacting with a single molecular target, cause the destabilization and eventual degradation of Hsp90 client proteins.

A second aspect of the present invention relates to novel medicaments and preparations comprising effective anti-cancer agents together with an anti-Hsp90 antibody which together provide an enhanced efficacy in the treatment of leukaemia.

Leukaemia is a cancer that affects the bone marrow. In people with leukaemia, the bone marrow produces large numbers of abnormal white blood cells. The abnormal white blood cells crowd into the bone marrow, so the marrow can't make enough normal red blood cells, white blood cells and platelets.

Different types of leukaemia can be categorised by their speed of development (acute or chronic), and by the type of white blood cell affected, (myeloid or lymphoid cells). Myeloid white blood cells are the immune system's first line of defense against infection and are found mainly in the blood, where they engulf and kill foreign organisms. Lymphoid white blood cells are found in the lymph nodes and in the blood.

The four most common types of leukaemia include chronic lymphoid (lymphocytic) leukaemia (CLL), acute myeloid (myeloblastic) leukaemia (AML), acute lymphoid (lymphoblastic) leukaemia (ALL), and chronic myeloid leukaemia (CML).

CLL is also a cancer of the lymphocyte cells but develops more slowly than ALL. This disease is the most common type of leukaemia affecting adults, and is very rare in children. AML is a cancer mainly affecting the myeloid cells known as granulocytes. It creates too many myeloblasts which can block blood vessels, and not enough mature myeloid cells. This disease occurs mainly in adults but can also affect children.

CML, (also called chronic granulocytic leukemia) is typically a slowly progressing cancer of the neutrophil cells, which is rare in children and commonly affects male adults more than females. CML is usually easily diagnosed because the leukaemic cells of more than 95% of patients have a distinctive cytogenetic abnormality, the Philadelphia chromosome (Ph1) (Kurzrock, R. et al. 2003, Ann. Intern. Med. 138 (10): p 819-30, PMID: 12755554; Goldman, J. M. and Melo, J. V., 2003, N. Engl. J. Med. 349 (15): p 1451-64, PMID: 14534339). The Ph1 results from a reciprocal translocation between the long arms of chromosomes 9 and 22 and is demonstrable in all haematopoietic precursors (Deininger, M. W. et al. 2000, Blood 96 (10): p 3343-56, PMID: 11071626). This translocation results in the transfer of the Abelson (abl) oncogene on chromosome 9, to an area of chromosome 22 termed the breakpoint cluster region (BCR) (Deininger, M. W. et al. 2000, Blood 96 (10): p 3343-56, PMID: 11071626). This in turn results in a fused BCR/ABL gene which encodes an 8.5 kb chimeric mRNA. The BCR/ABL gene is an oncogene which is sufficient to produce CML-like disease in mice. The transcript of the BCR/ABL oncogene is translated to yield a 210 kDa or 190 kDa protein. The Bcr-Abl protein is an abnormal tyrosine kinase protein that causes the disordered myelopoiesis found in CML. CML progresses through distinct clinical stages termed chronic phase, accelerated phase, and blast crisis. The BCR/ABL oncogene is expresses at all stages, but blast crisis is characterised by multiple additional genetic and molecular changes (Gorre, M. E., et al. 2002, Blood, 100(8): p 3041-3044).

Ph1-negative CML is a rare disease that is characterized by the clinical characteristics of CML without cytogenetic or molecular (RT-PCR) evidence of the t(9; 22)(q34; q11) translocation resulting in the Bcr-Abl fusion mRNA. Ph1-negative CML is a poorly defined entity that is less clearly distinguished from other myeloproliferative syndromes. Once thought to account for 5-10% of all clinical CML, with the routine accessibility of RT-PCR analysis for the Bcr-Abl transcript, that number is now well below 5%. Interestingly some patients with this entity may result from an alternative fusion to Abl. The TEL(ETV6)-ABL fusion, as a result of t(9; 12), has been demonstrated in two cases of Ph-CML. Patients with Ph1-negative CML generally have a poorer response to treatment and shorter survival than Ph1-positive patients (Onida, F. et al. 2002: Cancer 95 (8): p 1673-84, PMID: 12365015).

ALL is a cancer of immature lymphocyte cells, known as lymphoblasts. This disease is the most common type of leukaemia in young children, usually between the ages of 1 and 7 and is quite rare in adults. ALL causes many abnormal lymphocytes to be made, which crowd out the normal red blood cells and platelets. A 185 kDa Bcr-Abl protein has been directly implicated in the development in of ALL.

Two drugs, geldanamycin (GA), and 17-allylamino, 17-desmethoxygeldanamycin (17-AAG) which act as Hsp90 inhibitors, have showed promising biological and clinical activity in clinical trials. Indeed, the 210 kDa Bcr-Abl fusion protein (p210^{Bcr-Abl}) is dependent on its association with Hsp90 for its stability, and treatment of cells with GA or 17-AAG leads to rapid destruction of p210^{Bcr-Abl}.

An Hsp90 inhibitor such as 17AAG in combination with conventional cytotoxic agents or other novel agents, would also be therapeutically valuable in attacking multi step oncogenesis (Workman P., Cancer Lett. 2004 Apr. 8; 206(2):149-57; PMID: 15013520). In cancer cells, which are characterised by genetic instability, it is possible that 17AAG, by blocking Hsp90 activity, releases a variety of mutations that together prove “synthetically lethal” to the tumour. Normal cells, which lack the tumour cells' genetic instability, are relatively unaffected (Garber, K., 2002, Journal of the National Cancer Institute, Vol. 94, No. 22, p 1666-1668). A significant problem with 17AAG is that the drug is too toxic for prolonged therapy, and consequently there is a need for a non-toxic replacement (Banerji et al., supra).

Imatinib mesylate (Gleevec®) is a small molecule tyrosine kinase inhibitor that has had a major impact on a neoplastic disease as a single agent. Originally designed as an inhibitor of the Bcr-Abl tyrosine kinase characteristic of malignancies carrying the pathogenic 9; 22 translocation, Imatinib has proved to be moderately specific, and has made a major impact on the treatment of chronic myelogenous leukemia (CML) and Philadelphia chromosome positive (Ph1+) ALL (Krystal, G W, 2004, Leukemia Research 28S1:pS53-S59). One of the problems associated with imatinib treatment of CML, is resistance to the drug as a result of mutations in the Bcr-Abl tyrosine kinase. Importantly, CML cells that have become resistant to imatinib in vivo retain their Hsp90 dependence and thus remain sensitive to 17AAG.

Recent publications teach that tumour Hsp90 is present entirely in multi-chaperone complexes which facilitate malignant progression and that they are attractive targets for cancer therapeutics. In particular, Hsp 90 in multi-chaperone complexes derived from tumour cells is taught as having a 100-fold higher binding affinity for 17AAG than does Hsp90 from normal cells (i.e. Hsp90 in its latent uncomplexed state), indicating that in the multi-chaperone complex it may display epitopes (particularly quaternary epitopes) not displayed by the latent uncomplexed Hsp90. Mycograb® antibody can bind to Hsp 90 in its latent uncomplexed state, and also in multi-chaperone complexes, without any adverse effects on binding kinetics.

WO 01/76627 teaches compositions for treatment of fungal infections, the compositions comprising a combination of (i) a polyene or beta glucan synthase inhibitor antifungal agent; and (ii) antibodies specific against fungal Hsp90, the compositions being effective against the fungus causing the infection despite its being resistant to the antifungal agent per se.

According to a first aspect of the present invention there is provided the use of:
(i) an antibody or an antigen binding fragment thereof specific for at least one epitope of Hsp90; and
(ii) at least one anti-cancer agent selected from the group consisting of: Doxorubicin, Daunorubicin, Epirubicin, Herceptin, Docetaxel, and Cisplatin,
   in a method of manufacture of a medicament for the treatment of cancer.

Doxorubicin is an anthracycline antibiotic agent previously recognised as being an antitumour agent.

Epirubicin is a less toxic synthetic anthracyclin antibiotic, also previously recognised as being an antitumour agent.

Daunorubicin is an antineoplastic drug used in a number of therapeutic fields, including as an anti-cancer agent.

Herceptin (Trastuzumab) is a monoclonal antibody used for the treatment of HER2 protein overexpressing metastatic breast cancer.

Docetaxel is a recognised anti-cancer agent, and is a mitotic inhibitor.

Cisplatin is a recognised anti-cancer agent, and comprises a platinum complex.

As is detailed in the experimental results below (“Experiments A”), Doxorubicin and Daunorubicin are particularly preferred, and show particularly good synergistic effects with anti-Hsp90 antibody. Herceptin also shows good synergistic effects with anti-Hsp90 antibody. Synergy is also observed with Docetaxel and Cisplatin when combined with anti-Hsp90 antibody. The synergy between Daunorubicin and the antibody is particularly evident with oestrogen receptor positive cells, and so medicaments and therapies using the antibody and Daunorubicin may in particular be for (or administered to or for) cells having oestrogen receptors.

Experiments (“Experiments A”) also show that other anti-cancer agents when used together with anti-Hsp90 antibody either show indifferent results (Paclitaxel) or antagonism (Imatinib). This confirms the surprising/unexpected nature of the synergy achieved with the above anti-cancer agents when combined with anti-Hsp90 antibody.

Also provided is a combined preparation comprising:
(i) an antibody or an antigen binding fragment thereof specific for at least one epitope of Hsp90; and
(ii) at least one anti-cancer agent selected from the group consisting of: Doxorubicin, Daunorubicin, Epirubicin, Herceptin, Docetaxel, and Cisplatin,
   for simultaneous, separate or sequential use in the treatment of cancer.

Also provided is a method of treatment of cancer comprising administering a therapeutically effective quantity of:
(i) an antibody or an antigen binding fragment thereof specific for at least one epitope of Hsp90; and
(ii) at least one anti-cancer agent selected from the group consisting of: Doxorubicin, Daunorubicin, Epirubicin, Herceptin, Docetaxel, and Cisplatin,
   to a patient in need of same.

As used herein, the term “treatment” is intended to have a broad meaning unless explicitly stated otherwise. Thus by “treatment” or “therapy” is meant any treatment which is designed to cure, alleviate, remove or lessen the symptoms of, or prevent or reduce the possibility of contracting disorders or malfunctions of the human or animal body. Thus by the term “treatment” is meant both treatment of disease conditions, as well as their prophylaxis.

The antibody or antigen binding fragment thereof may be specific for the epitope displayed by a peptide comprising the sequence of SEQ ID NO: 1.

As discussed above, although quaternary epitopes displayed by Hsp90 in multi-chaperone complexes have been suggested as appropriate targets for therapy, experiments (below) show that in fact a linear epitope is a useful and effective target for therapy.

Antibodies, their manufacture and uses are well known and disclosed in, for example, Harlow, E. and Lane, D., Antibodies: A Laboratory Manual, Cold Spring Harbor Laboratory Press, Cold Spring Harbor, N.Y., 1999.

The antibodies may be generated using standard methods known in the art. Examples of antibodies include (but are not limited to) polyclonal, monoclonal, chimeric, single chain, Fab fragments, fragments produced by a Fab expression library, and antigen binding fragments of antibodies.

Antibodies may be produced in a range of hosts, for example goats, rabbits, rats, mice, humans, and others. They may be immunized by injection with fungal stress proteins, or any fragment or oligopeptide thereof which has immunogenic properties. Depending on the host species, various adjuvants may be used to increase an immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. Among adjuvants used in humans, BCG (Bacille Calmette-Guerin) and Corynebacterium parvum are particularly useful.

Monoclonal antibodies to fungal proteins, or any fragment or oligopeptide thereof may be prepared using any technique which provides for the production of antibody molecules by continuous cell lines in culture. These include, but are not limited to, the hybridoma technique, the human B-cell hybridoma technique, and the EBV-hybridoma technique (Koehler et al., 1975, Nature, 256: 495-497; Kosbor et al., 1983, Immunol. Today 4: 72; Cote et al., 1983, PNAS USA, 80: 2026-2030; Cole et al., 1985, Monoclonal Antibodies and Cancer Therapy, Alan R. Liss Inc., New York, pp. 77-96).

In addition, techniques developed for the production of “chimeric antibodies”, the splicing of mouse antibody genes to human antibody genes to obtain a molecule with appropriate antigen specificity and biological activity can be used (Morrison et al., 1984, PNAS USA, 81: 6851-6855; Neuberger et al., 1984, Nature, 312: 604-608; Takeda et al., 1985, Nature, 314: 452-454). Alternatively, techniques described for the production of single chain antibodies may be adapted, using methods known in the art, to produce fungal stress protein-specific single chain antibodies. Antibodies with related specificity, but of distinct idiotypic composition, may be generated by chain shuffling from random combinatorial immunoglobin libraries (Burton, D. R., 1991, PNAS USA, 88:11120-11123).

Antibodies may also be produced by inducing in vivo production in the lymphocyte population or by screening recombinant immunoglobulin libraries or panels of highly specific binding reagents (Orlandi et al., 1989, PNAS USA, 86: 3833-3837; Winter, G. et al., 1991, Nature, 349: 293-299).

Antigen binding fragments may also be generated, for example the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed to allow rapid and easy identification of monoclonal Fab fragments with the desired specificity (Huse et al., 1989, Science, 256: 1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between the fungal stress protein or any fragment or oligopeptide thereof, and its specific antibody. A two-site, monoclonal-based immunoassay utilizing monoclonal antibodies specific to two non-interfering fungal stress protein epitopes may be used, but a competitive binding assay may also be employed (Maddox et al., 1983, J. Exp. Med., 158: 1211-1216).

For example, the antibody used in the composition or combined preparation may comprise the sequence of SEQ ID NO: 2.

The present inventor has found that cancers which may be usefully treated include fibrosarcomas and carcinomas selected from the group consisting: breast, prostate, melanoma, leukemia, lymphomas, leukemia, colon, testicular germ cell, pancreatic, ovarian, endometrial, thyroid, and lung.

According to a second aspect of the present invention there is provided the use of:
(i) an antibody or an antigen binding fragment thereof specific for at least one epitope of Hsp90;
   in a method of manufacture of a medicament for the treatment of leukaemia.

Also provided is the use of:
(i) an antibody or an antigen binding fragment thereof specific for at least one epitope of Hsp90; and
   (ii) at least one anti-cancer agent selected from the group consisting of: Imatinib, Paclitaxel, Docetaxel, Daunorubicin, Doxorubicin, and Hydroxyurea,
      in a method of manufacture of a medicament for the treatment of leukaemia.

Imatinib, a derivative of 2-phenylaminopyrimidine, is a small molecule antagonist with activity against protein tyrosine kinases, and exhibits potent and specific inhibition of Bcr-Abl. Imatinib is indicated for the treatment of patients with CML in blast crisis, accelerated phase, or in chronic phase after failure of IFN-therapy.

Paclitaxel is chemotherapeutic agent that is given as a treatment for some types of cancer. It is most commonly used to treat ovarian, breast and non-small cell lung cancer.

Docetaxel is a recognised anti-cancer agent, and is a mitotic inhibitor.

Daunorubicin is an anti-neoplastic drug used in a number of therapeutic fields, including as an anti-cancer agent.

Doxorubicin is an anthracycline antibiotic agent previously recognised as being an anti-tumour agent.

Hydroxyurea is an anti-neoplastic, ribonucleotide reductase inhibitor.

As is detailed in the experimental results below (“Experiments B”), Doxetaxel and Paclitaxel are particularly preferred, and show particularly good synergistic effects with anti-Hsp90 antibody. Synergy is also observed with Imatinib, Doxorubicin, Daunorubicin, and Hydroxyurea when combined with anti-Hsp90 antibody. The anti-cancer agent Cisplatin, when used together with anti-Hsp90 antibody showed indifferent results. This confirms the surprising/unexpected nature of the synergy achieved with the above anti-cancer agents when combined with anti-Hsp90 antibody.

Also provided is a combined preparation comprising:
(i) an antibody or an antigen binding fragment thereof specific for at least one epitope of Hsp90; and
(ii) at least one anti-cancer agent selected from the group consisting of: Imatinib, Paclitaxel, Docetaxel, Daunorubicin, Doxorubicin, and Hydroxyurea,
   for simultaneous, separate or sequential use in the treatment of leukaemia.

Examples of combined preparations include pharmaceutical packs containing the antibody of (i) and at least one anti-cancer agent of (ii) in separate volumes (i.e. not mixed together in a single preparation).

Also provided is a method of treatment of leukaemia comprising administering a therapeutically effective quantity of:
(i) an antibody or an antigen binding fragment thereof specific for at least one epitope of Hsp90; and
(ii) at least one anti-cancer agent selected from the group consisting of: Imatinib, Paclitaxel, Docetaxel, Daunorubicin, Doxorubicin, and Hydroxyurea,
   to a patient in need of same.

The leukaemia may be chronic myeloid leukaemia or acute lymphoid leukaemia, and the at least one anti-cancer agent may Imatinib.

The antibody or antigen binding fragment thereof may be specific for the epitope displayed by a peptide comprising the sequence of SEQ ID NO: 1.

For example, the antibody used in the composition or combined preparation may comprise the sequence of SEQ ID NO: 2.

The anti-cancer agent may be Imatinib.

The present inventor has found that leukaemias which may be usefully treated include leukaemias selected from the group consisting of: acute myeloblastic leukaemia, acute lymphoblastic leukaemia, chronic myeloid leukaemia, and chronic lymphocytic leukaemia.

The leukaemia may be chronic myeloid leukaemia or acute lymphoid leukaemia.

The chronic myeloid leukaemia may be Ph1-positive or Ph1-negative, i.e is characterized by leukaemic cells which contain the Philadelphia chromosome (Ph1-positive), or lack the Philadelphia chromosome (Ph1-negative).

The present inventor has found that chronic myeloid leukaemias which may be usefully treated with Imatinib may be either Ph1-positive or Ph1-negative.

The leukaemia may be chronic myeloid leukaemia which is Ph1-positive, and the anti-cancer agent may be Imatinib. In particular, the present inventor has found that treatment of CML which is Ph1-positive can be effected by a combination of Imatinib and an antibody comprising the sequence of SEQ ID NO: 2. Without wishing to be bound by any theory, it is possible that Hsp90 is sequestered by the antibody comprising the sequence of SEQ ID NO: 2, which in turn means that the abnormal Bcr-Abl tyrosine kinase (which causes the disordered myelopoiesis found in CML) is e.g. incorrectly folded, targeted for protein degradation, and/or prevented from exerting it's effects on myelopoietic pathways.

This treatment is further effective on Imatinib resistant CML Ph1-positive cells. Without wishing to be bound by any theory, the resistance to Imatinib is likely to be due to collected mutations in the abnormal Bcr-Abl tyrosine kinase which could e.g. prevent the drug from binding to the protein and/or interfere with the mode of action of the drug. In Imatinib resistant cells, it is possible that the sequestration of Hsp90 by the antibody comprising the sequence of SEQ ID NO: 2, causes the mutated abnormal tyrosine kinase to be e.g. incorrectly folded, targeted for protein degradation, and/or prevented from exerting it's effects on myelopoietic pathways. The sequestration of Hsp90, which normally serves to “buffer” the genetic mutations associated with cancerous cells by binding to abnormal proteins and blocking their expression, may also cause a variety of mutations to be released which together prove synthetically lethal to the tumour cell. Normal cells, which lack the tumour cells' genetic instability, are relatively unaffected.

Additionally, and particularly surprisingly, the present inventor has found that treatment of CML which is Ph1-negative can be effected by a combination of Imatinib and an antibody comprising the sequence of SEQ ID NO: 2.

The leukaemia may be chronic myeloid leukaemia which is Ph1-negative, and the anti-cancer agent may be Imatinib.

This finding is surprising because Ph1-negative CML cells lack the abnormal tyrosine kinase protein associated with Ph1-positive cells. However, and without wishing to be bound by any theory, it is possible there are low or basal levels of this kinase (whether abnormal or otherwise) in Ph1-negative cells, and as described above, the sequestration of Hsp90 is by the antibody comprising the sequence of SEQ ID NO: 2, means that the tyrosine kinase protein is e.g incorrectly folded, or targeted for protein degradation, or in some way prevented from exerting its effects on myelopoietic pathways. It may also be the case that by sequestering Hsp90, a variety of mutations are released by the tumour cell which together prove synthetically lethal.

The surprising effect of Imatinib and the anti-Hsp90 antibody in Ph1-negative cells may be due to the presence of the TEL(ETV6)-ABL fusion, which has been demonstrated in two cases of Ph1-negative CML (Krystal, G W, 2004, Leukemia Research 28S1:pS53-S59), and which is sensitive to Imatinib.

The leukaemia may be characterised by cells which are Imatinib resistant.

The composition or preparation of the present invention may additionally comprise a known Hsp 90 inhibitor, for example GA, or 17-AAG.

A third aspect of the present invention (Experiments C) relates to novel medicaments and preparations comprising effective anti-cancer agents together with anti-Hsp90 antibody which together provide an enhanced efficacy in the treatment of colorectal cancer or adenocarcinomas.

Colorectal cancer is a malignant tumour of the colon or rectum. Colorectal cancer is a leading cause of cancer morbidity and mortality. It is the third most common cancer in men and the second most common cancer in women in the UK. Ninety five percent of colorectal cancers are adenocarcinomas, which are cancers of the glandular call that line the inside of the colon and rectum.

Standard treatment of colorectal cancer is usually a combination of 5-fluorouracil and leucovorin (folinic acid).

5-fluorouracil (5-FU) is used to treat a number of solid tumours, including gastro-intestinal cancers and breast cancer. It is commonly used with folinic acid in advanced colorectal cancer. 5-FU is converted to FdUMP in the cell, which forms a complex with Thymidylate synthase (TS) inhibiting DNA, protein and RNA synthesis.

Folinic acid (Leucovorin) is a vitamin which is given in combination with 5-FU. Folinic acid increases the response rate to 5-fluorouracil, with a significant improvement in disease free and overall survival. Folinic acid increases the intracellular folate and stabilises the FdUMP/TS complex.

Other agents found to have an effect include irinotecan and oxalipatin, which is licensed for first-line use in patients with advanced colorectal cancer, in combination with 5-fluorouracil and folinic acid. Irinotecan or raltitrexed are licensed for use as a second-line monotherapy when fluorouracil-based therapy has failed or is inappropriate.

Oxaliplatin is a recognised anti-cancer agent and contains a novel diaminocyclohexane platinum compound which forms cross-links in DNA and so inhibits DNA replication.

‘FOLFOX’ is the commonly used combination chemotherapy of 5-fluorouracil, folinic acid and Oxaliplatin.

Irinotecan (CPT-11, Campto) inhibits topoisomerase I, a DNA-unwinding enzyme essential for cell division, which results in replication arrest with breaks in single-strand DNA. In the UK, irinotecan is licensed for use in chemotherapy-naïve patients with advanced colorectal cancer in combination with 5FU/FA and as a single agent for second-line chemotherapy in patients who have failed an established 5FU-based regimen.

Raltitrexed (ZD 1694, Tomudex) inhibits the enzyme thymidylate synthetase, which is involved in DNA synthesis. This is the same enzyme that is targeted by 5FU. Raltitrexed is licensed in the UK for the palliative treatment of advanced colorectal cancer where 5FU/FA-based regimens are either not tolerated or inappropriate.

Tebbutt et al., 2002, European Journal of Cancer, 38: 1000-1015; Cutsem et al., 2002, Best Practice and Research Clinical Gastroenterology, 16: 319-330; Beretta et al., 2004, Surgical Oncology, 13: 63-73; NICE guidelines for Irinotecan, Oxaliplatin and raltitrexed for advanced colorectal cancer, 2002.

According to this third aspect of the invention, there is provided the use of:
(i) an antibody or an antigen binding fragment thereof specific for at least one epitope of Hsp90; and
(ii) at least one anti-cancer agent selected from the group consisting of: 5-fluorouracil, oxaliplatin, irinotecan and raltitrexed, in a method of manufacture of a medicament for the treatment of cancer.

Alternatively, there is provided a combined preparation comprising:
(i) an antibody or an antigen binding fragment thereof specific for at least one epitope of Hsp90; and
(ii) at least one anti-cancer agent selected from the group consisting of: 5-fluorouracil, oxaliplatin, irinotecan and raltitrexed,
   for simultaneous, separate or sequential use in the treatment of cancer.

According to yet a further aspect of this invention, there is provided a method of treatment of cancer comprising administering a therapeutically effective quantity of:
(i) an antibody or an antigen binding fragment thereof specific for at least one epitope of Hsp90; and
(ii) at least one anti-cancer agent selected from the group consisting of: 5-fluorouracil, oxaliplatin, irinotecan and raltitrexed,
   to a patient in need of same.

Preferably, the cancer is colorectal cancer or adenocarcinoma.

Most preferably, the anti-cancer agent 5-fluorouracil further comprises or is administered with folonic acid (leucovorin).

Additionally or alternatively, 5-fluorouracil, folinic acid and oxaliplatin are administered together.

The composition or preparation according to any aspect of this invention may additionally comprise a pharmaceutically acceptable carrier, diluent or excipient. Similarly, any method of manufacture of the present invention or use in same may also comprise the use of a pharmaceutically acceptable carrier, diluent or excipient. Examples of pharmaceutically acceptable carriers, diluents and excipients are well known in the art, for example see: Remington's Pharmaceutical Sciences and US Pharmacopoeia, (1984, Mack Publishing Company, Easton, Pa., USA).

The medicaments or combined preparation may, for example, be administered orally although this does not mean that other administration routes are to be excluded.

The antibody or antigen binding fragment thereof according to the present invention may be labelled with a detectable label or may be conjugated with an effector molecule, for example a drug e.g. an anti-cancer agent such as Doxorubicin, Daunorubicin, Docetaxel, or Cisplatin, or 5-fluorouracil, oxaliplatin, irinotecan and raltitrexed or a pharmaceutical agent useful in treating leukaemia e.g. Imatinib, Paclitaxel, Docetaxel, Daunorubicin, Doxorubicin, and Hydroxyurea, or a toxin, such as ricin, or an enzyme, using conventional procedures, and the invention extends to such labelled antibodies or antibody conjugates.

If desired, mixtures of antibodies may be used for diagnosis or treatment, for example mixtures of two or more antibodies recognising different epitopes of a stress protein according to the invention, and/or mixtures of antibodies of a different class, e.g. mixtures of IgG and IgM antibodies recognising the same or different epitope(s) of the invention.

As discussed above, although quaternary epitopes displayed by Hsp90 in multi-chaperone complexes have been suggested as appropriate targets for therapy, experiments (below) show that in fact a linear epitope is a useful and effective target for therapy.

The contents of each of the references discussed herein, including the references cited therein, are herein incorporated by reference in their entirety.

Where “PMID:” reference numbers are given for publications, these are the PubMed identification numbers allocated to them by the US National Library of Medicine, from which full bibliographic information and abstract for each publication is available at www.ncbi.nlm.nih.gov. This can also provide direct access to electronic copies of the complete publications, particularly in the case of e.g. PNAS, JBC and MBC publications.

The present invention will be further apparent from the following description, which shows, by way of example only, specific embodiments of the composition and experimentation therewith.

### EXPERIMENTS

A first set of experiments (“Experiments A”) described below detail the investigation of the anti-cancer effect of an anti-Hsp90 antibody having the sequence of SEQ ID NO: 2 and specific for an epitope displayed by a peptide having the sequence of SEQ ID NO: 1, used on its own or in combination with the anti-cancer agents Doxorubicin, Daunorubicin, Docetaxel, Herceptin, Imatinib, Cisplatin, and Paclitaxel.

A second set of experiments (“Experiments B”) described below detail the investigation of the effect of an anti-Hsp90 antibody having the sequence of SEQ ID NO: 2 and specific for an epitope displayed by a peptide having the sequence of SEQ ID NO: 1, used on its own or in combination with the anti-cancer agents Imatinib, Paclitaxel, Docetaxel, Daunorubicin, Doxorubicin, Paclitaxel, Cisplatin, and Hydroxyurea, on the human Caucasian chronic myelogenous leukaemia cell line K562, and human myelogenous leukaemia cell line KU-812.

A third set of experiments, (“Experiments C”) describe below in detail the investigation of the effect of an anti-Hsp90 antibody having the sequence of SEQ ID NO: 2 and specific for epitope displayed by a peptide having the sequence of SEQ ID NO: 1, used on its own or in combination with the anti-cancer agent's 5-Fluorouracil (5-FU) and Folinic acid (Leucovorin, LV) and/or Oxaliplatin on the human colon adenocarcinoma cell line HT29.

General Materials and Methods

Unless stated otherwise, all procedures were performed using standard protocols and following manufacturer's instructions where applicable. Standard protocols for various techniques including PCR, molecular cloning, manipulation and sequencing, the manufacture of antibodies, epitope mapping and mimotope design, cell culturing and phage display, are described in texts such as McPherson, M J et al. (1991, PCR: A practical approach, Oxford University Press, Oxford), Sambrook, J. and Russell, D., “Molecular Cloning: A Laboratory Manual”, Third Edition, Cold Spring Harbor Laboratory, Cold Spring Harbor Press, New York, 2001, Huynh and Davies (1985, “DNA Cloning Vol I—A Practical Approach”, IRL Press, Oxford, Ed. DM Glover), Sanger, F. et al. (1977, PNAS USA 74(12): 5463-5467), Harlow, E. and Lane, D. (“Using Antibodies: A Laboratory Manual”, Cold Spring Harbor Laboratory Press, New York, 1998), Jung, G. and Beck-Sickinger, AG (1992, Angew. Chem. Int. Ed. Eng., 31: 367-486), Harris, M. A. and Rae, I. F. (“General Techniques of Cell Culture”, 1997, Cambridge University Press, ISBN 0521 573645), “Phage Display of Peptides and Proteins: A Laboratory Manual” (Eds. Kay, B K, Winter, J., and McCafferty, J., Academic Press Inc., 1996, ISBN 0-12-402380-0).

Reagents and equipment useful in, amongst others, the methods detailed herein are available from the likes of Amersham (www.amersham.co.uk), Boehringer Mannheim (www.boehringer-ingeltheim.com), Clontech (www.clontech.com), Genosys (www.genosys.com), Millipore (www.millipore.com), Novagen (www.novagen.com), Perkin Elmer (www.perkinelmer.com), Pharmacia (www.pharmacia.com), Promega (www.promega.com), Qiagen (www.qiagen.com), Sigma (www.sigma-aldrich.com) and Stratagene (www.stratagene.com).

Antibody

The antibody used in Experiments A and B below is that disclosed in WO 01/76627, and is herein referred to as Mycograb®, having the sequence of SEQ ID NO: 2 and being specific for an epitope displayed by the peptide having the sequence of SEQ ID NO: 1. The basic antibody solution was a 4 mg/ml stock solution in water. Further dilutions were carried out in RPMI complete medium.

Briefly, the DNA sequence of a former antibody specific for the Candida albicans Hsp90 epitope disclosed in GB 2240979 and EP 0406029 was genetically modified by codon optimisation for expression in Escherichia coli (Operon Technologies Inc., Alameda, Calif., USA) and inserted into an E. coli expression vector. The amino acid sequence of the anti-Hsp90 antibody comprises the sequence of SEQ ID NO: 2 (includes the heavy, light and spacer domains). The antibody recognises the epitope comprising the sequence of SEQ ID NO: 1.

The anti-Hsp90 antibody was expressed in an Escherichia coli host and then purified by affinity chromatography and an imidazole exchange column up to 95% purity. Standard molecular biology protocols were employed (see, for example, Harlow & Lane, supra; Sambrook, J. et al., 1989, Molecular Cloning: A Laboratory Manual, 2nd Edition, Cold Spring Harbor Laboratory Press, Cold Spring Harbor, N.Y.; Sambrook, J. & Russell, D., 2001, Molecular Cloning: A Laboratory Manual, 3rd Edition, Cold Spring Harbor Laboratory Press, Cold Spring Harbor).

Drugs

Cisplatin was obtained from Bristol-Myers Squibb, Mayne, supplied as 1 mg/ml.

Docetaxel was obtained from Sigma. 5 mg was diluted initially to 16 mg/ml with Dimethyl sulfoxide (DMSO).

Doxorubicin was obtained from Pharmacia; 5 ml supplied as Doxorubicin hydrochloride 2 mg/ml.

Imatinib (Glivec®), obtained from Novartis, was supplied as 100 mg capsule. Imatinib was initially diluted in water to produce a 10 mg/ml stock solution.

Pacilitaxel was obtained from Sigma, reconstituted in 250 μl methanol made up to 2.5 ml with water to give 2 mg/ml.

Daunorubicin was obtained from Sigma, 5 mg was diluted in 2.5 ml water to give 2 mg/ml.

Herceptin® (Trastuzumab) was obtained from Roche and reconstituted in 7.2 ml of water to give 21 mg/ml.

Hydroxyurea was obtained from Sigma, with 1 g diluted in 4 ml water to give 25 mg/ml.

5-Fluorouracil (5-FU) was obtained from Sigma, 96 mg was reconstituted in 1 ml DMSO diluted 1/10 in complete RPMI media to give 9.6 mg/ml.

Folinic acid (LV) was obtained from Sigma; 100 mg was reconstituted in 25 ml of water to give a 4 mg/ml stock solution.

Oxaliplatin was obtained from Sigma; 12.5 mg was reconstituted in 2.5 ml of water to give 5 mg/ml.

Each of the above drugs was further diluted in RPMI complete medium.

Cell Concentration and Viability Determination

Cells were counted and percentage viability determined using a standard haemocytometer following staining with an equal volume of 0.4% Trypan Blue solution (Sigma).

Cell Viability Assay

Cell viabilities were assessed after each experiment using the Cell Titer Blue Assay (Promega). Media was removed from the cells and 100 μl of fresh complete medium added followed by 20 μl of Cell Titer Blue Reagent. This was incubated at 37° C., 5% CO₂ for 4 hours and absorbance read at 570 nm using 600 nm as a reference. This assay uses the indicator dye resazurin (blue) to measure the metabolic capacity of the cells. Viable cells reduce resazurin to resorufin (pink).

Data Interpretation

Cell growth was evaluated as described above. The IC₅₀ (the dose of drug needed to cause cytotoxicity in 50% of the cells) concentrations were determined singly for each drug over 48 hour incubation periods.

Median effect analysis, a measure of synergism, additive effects, or antagonism based upon the Hill equation, was determined by the method of Chou and Talalay using the Calcusyn product (BioSoft, Cambridge, UK—www.biosoft.com). The CI (combination index) which reflects synergy when less than 1, additive effects when equal to 1, and antagonism when greater than 1 was calculated for varying levels of drug effect. Ten fixed drug ratios above and below the IC₅₀ (the concentration of drug required to exert a 50% cytotoxic effect) with a range of 0.0156N-8N where N is a value near the IC₅₀ of an individual drug were explored by incubating the drug combinations with cells for 48 hours and then determining the degree of cytotoxicity. Fa50 is defined at that point where 50% of the cells are affected. CI values are shown for Fa50.

### Experiments A

The results show that the antibody gave evidence of antagonism with Imatinib and indifference with Paclitaxel. There was some synergy with Cisplatin and with Docetaxel, but the latter is probable at concentrations which cannot be achieved clinically. Doxorubicin demonstrated synergy at clinically achievable drug levels with both cell lines and independently of whether there was an oestrogen receptor. The results achieved with Doxorubicin rate as a highly significant synergy. The results of Daunorubicin were equally impressive with the cell line with an oestrogen receptor but less with the oestrogen receptor negative cell line with synergy restricted to 6 and 12.5 mg/l. The results for Herceptin showed Mycograb (1.5-200 μg/ml) or formulation buffer or medium alone was then added to the wells. The plate was returned to the incubator for 48 hours following which the cell titre blue assay was carried out or viable counts were carried out using a haemocytometer.

Effect of Anti-cancer Agents Doxorubicin, Daunorubicin, Herceptin, Docetaxel, Imatinib, Paclitaxel and Cisplatin on MCF7 Cells

The cell lines were split and cells counted. 100 μl of 4×10⁴ cells/ml were added to 96 well flat bottomed tissue culture plates a further 100 μl of complete medium was added to the plate. The plates were then incubated overnight at 37° C., 5% CO₂. The next day, the cells were observed under phase contrast microscopy to ensure they had adhered to the plates and the supernatant medium removed by aspiration. Fresh medium containing increasing concentrations of study drug (Doxorubicin 0.55-600 μg/ml, Daunorubicin 0.45-1000 μg/ml, Herceptin 0.2-200 μg/ml, Docetaxel 0.75-800 μg/ml, Imatinib 4.5-5000 μg/ml, Cisplatin 0.04-50 μg/ml, Paclitaxel 1.8-1000 μg/ml) or medium alone was added to the wells. The plates were returned to the incubator for 48 hours following which cell titre blue assays were carried out.

Effect of Mycograb in Combination with Anti-Cancer Agents Doxorubicin, Daunorubicin, Herceptin, Docetaxel, Imatinib, Paclitaxel and Cisplatin on MCF7 Cells

The cell lines were split and cells counted. 100 μl of 4×10⁴ cells/ml were added to 96 well flat bottomed tissue culture plates a further 100 μl of complete medium was added to the plate. The plates were then incubated overnight at 37° C., 5% CO₂. The next day, the cells were observed under phase contrast microscopy to ensure they had adhered to the plates and the supernatant medium removed by aspiration. 100 μl of fresh medium was added to the plates and a checkerboard of Mycograb versus other drug was set out as outlined in Table 1 below (using Doxorubicin as an example) to give a total volume of 200 μl per well.

The plates were returned to the incubator for 48 hours following which cell titer blue assays were carried out.

Experiments were also performed with HS578T cells using the above methodologies, and results are given below.

no synergy against an oestrogen receptor positive cell line, but synergy was observed with an oestrogen receptor negative cell line.

Materials and Methods

Cell Line and Culture Information

Human Caucasian breast adenocarcinoma cell line MCF7, expressing both wild type and variant oestrogen receptors as well a progesterone receptor, was obtained from ECACC (ECACC number—86012803).

Other cell lines used are as follows:

HS578T—ECACC number 86082104, Human breast carcinoma, Epithelial. Tumorigenic in immunosuppressed mice and form colonies in semisolid medium. Oestrogen receptor negative.

SK-BR-3—(ATCC) Human breast adenocarcinoma. Oestrogen receptor positive. Over expresses HER2/C-erb-2 gene.

UACC-812—(ATCC) Ductal Carcinoma, prior to surgery, patient had extensive chemotherapy. Oestrogen receptor negative, progesterone receptor negative, P-glycoprotein negative. Amplification of HER-2/neu oncogene sequence

HCT116—ATCC Colorectal carcinoma. Positive for TGF Beta 1 and beta 2 expression

Cells were split using 0.25% trypsin/EDTA (Sigma) and maintained in RPMI medium without phenol red, containing 10% Foetal Bovine Serum, 1% Non Essential Amino Acids, 2 mM Glutamine, 100 U/ml Penicillin, 0.1 mg/ml Streptomycin (Sigma) at 37° C., 5% CO₂.

Experiments A

Effect of Mycograb on MCF7 Cells

The cell lines were split and cells counted. Cells were added to 12- or 96-well flat-bottomed tissue culture plates. In the case of the 12-well plates, 1 ml of 4×10⁴ cells/ml were added plus 1 ml of medium. In the case of the 96-well plate, 100 μl of 4×10⁴ cells/ml were added followed by a further 100 μl of complete medium was added to the plate. The plates were incubated overnight at 37° C., 5% CO₂. The next day, the cells were observed under phase contrast microscopy to ensure they had adhered to the plates and the supernatant medium removed by aspiration. Fresh medium containing twofold increasing concentrations of

Results of Experiments A

MCF7 Cell Line

Cisplatin

The IC₅₀ was 6 mg/l and there was no effect on adding Mycograb with the exception of synergy at higher concentrations—see Table 2.

Imatinib

The IC₅₀ was 37.5 mg/l and there was evidence of antagonism with CIs in the range of 3.3-10 with Mycograb at doses of Imatinib below 37.5 mg/l. Above this dose the Imatinib killed the cell line.

Docetaxel

The IC50 was 225 mg/l and there was evidence of some synergy with Mycograb at high doses of Docetaxel—see Table 3.

Paclitaxel

The IC₅₀ was 225 mg/l and there was indifference with low concentrations of the drug and mild synergy at high levels such as 500 mg/l of Paclitaxel. These levels are outside those that are clinically relevant.

Doxorubicin

The IC₅₀ was 1.75 mg/l. There was clear synergy with Mycograb over a range of drug concentrations—see Table 4.

Daunorubicin

The IC₅₀ was 1 mg/l. There was evidence of synergy with Mycograb over a range of drug concentrations—see Table 5.

Herceptin

There was no detectable activity due to Herceptin and no evidence of synergy.

HS578T Cell Line

This cell line was insensitive to Mycograb in increasing concentrations up to 400 mg/l. This was not surprising in that these tumours are not steroid sensitive and thus not intrinsically likely to respond to an Hsp90 inhibitor such as Mycograb. However in combination with the anthracycline Doxorubicin, as well as Daunorubicin, and Herceptin there was unexpected synergy.

Doxorubicin

The IC50 was 1 mg/l. There was evidence of synergy with Mycograb over a range of drug concentrations—see Table 6.

Daunorubicin

The IC₅₀ was 1 mg/l. There was some evidence of synergy but mostly indifference with Mycograb—see Table 7.

Herceptin

With HS578T, mono-herceptin failed to kill 50% of the cells in concentrations of up to 200 mg/l, but in the presence of Mycograb synergy was observed—see Table 8.

Docetaxel

This gave an IC₅₀ of 50 mg/l for the cell line HS578T and showed no evidence of synergy with Mycograb.

Cisplatin

Cisplatin had an IC₅₀ of 12.5 mg/l for the cell line HS578T and showed no evidence of synergy with Mycograb

Conclusion

There was evidence of antagonism with Imatinib and indifference with Paclitaxel. There was some synergy with Cisplatin and with Docetaxel, but the latter is probable at concentrations which cannot be achieved clinically. Doxorubicin demonstrated synergy at clinically achievable drug levels with both cell lines and independently of whether there was an oestrogen receptor. The results achieved with Doxorubicin rate as a highly significant synergy. The results of Daunorubicin were equally impressive with the cell line with an oestrogen receptor but less with the oestrogen receptor negative cell line with synergy restricted to 6 and 12.5 mg/l.

This above surprising synergistic effect is observed between the antibody and certain anti-cancer drugs, but not with other anti-cancer agents such as Imatinib.

### Experiments B

A second set of experiments (described below) detail the investigation of the effect of an anti-Hsp90 antibody having the sequence of SEQ ID NO: 2 and specific for an epitope displayed by a peptide having the sequence of SEQ ID NO: 1, used on its own or in combination with the anti-cancer agents Imatinib, Paclitaxel, Docetaxel, Daunorubicin, Doxorubicin, Paclitaxel, Cisplatin, and Hydroxyurea, on the human Caucasian chronic myelogenous leukaemia cell line K562, and human myelogenous leukaemia cell line KU-812.

The results show that the antibody gave evidence of synergy with Imatinib, Paclitaxel, Docetaxel, Daunorubicin. The antibody gave evidence of some synergy with Doxorubicin and Hydroxyurea. The results show that the antibody gave evidence of indifference with Cisplatin.

The results achieved with Docetaxel and Paclitaxel rate as a highly significant synergy.

Material and Methods

Cell Line and Culture Information

Human myelogenous leukaemia cell line KU-812 was obtained from ECACC (ECAAC number 90071807). A Philadelphia chromosome (Ph1) has been detected in this cell line. The cells are morphologically characteristic of basophils.

Human Caucasian chronic myelogenous leukaemia cell line K562, was obtained from ECACC (ECACC number 89121407). K562 was established from pleural effusion of 53 year old female with chronic myelogenous leukaemia in terminal blast crisis. Karyological studies on various K-562 sublines have been classified into three groups (A, B, C) (Dimery, I. W. et al., 1983, Exp. Hematol.; 11(7):p 601-10). The line used in these experiments was the K562B. Experiments have demonstrated that these lines are generally similar in terms of: morphology, growth kinetics in liquid suspension culture, cloning efficiency in soft agar culture, binding of anti-K562 monoclonal antibodies, and cell surface proteins. K562B has been compared to K562A and K562 C, with respect to growth kinetics, cell surface protein markers, surface antigens, cytogenetics and hemoglobin production. Differences were observed between the cell lines, the most important difference being that whereas more than 90% of K562A or C cells appeared to be Ph1-positive, less than 15% of K562B cells contained a Ph1 (Dimery, I W, et al., 1983, Exp. Hematol.; 11(7):p 601-10). Although cytogenetic tests do not reveal the presence of a true Ph1 chromosome K562 appears to contain part of a Ph1 chromosome, which is at least fourfold amplified. This part of a Ph1 chromosome encodes a chimeric bcr/c-abl transcript, which when translated yields a bcr/c-abl fusion protein (Grosveld, G., et al., 1986, Mol. Cell. Biol. 6, No. 2: p 607-616). The bcr/c-abl fusion protein possesses activated tyrosine kinase activity which is responsible for the pathogenesis of CML.

Cells were maintained between 2×10⁶ and 9×10⁶ cell/ml in RPMI medium 1640 without phenol red, containing 10% Foetal Bovine Serum, 2 mM Glutamine, 100 U/ml Penicillin, 0.1 mg/ml Streptomycin (Sigma) at 37° C., 5% CO₂.

Experiments

Effect of Mycograb on K562 Cells

The cell lines were counted. Cells were added to 96 well flat-bottomed tissue culture plates using aliquots of 100 μl containing 4×10⁵ cells/ml. Fresh medium containing either two-fold increasing concentrations of Mycograb® (1.5-200 μg/ml), or medium alone was then added to the wells. The plate was returned to the incubator for 48 hours following which the cell titre blue assay was carried out, or viable counts were determined using a haemocytometer.

Effect of anti-cancer agents Doxorubicin, Daunorubicin, Docetaxel, Paclitaxel, Imatinib, Cisplatin and Hydroxyurea on K562 Cells

The cell lines were counted. 100 μl of 2×10⁵ or 4×10⁵ cells/ml were added to 96 well flat bottomed tissue culture plates. The plates were then incubated overnight at 37° C., 5% CO₂. Fresh medium containing increasing concentrations of study drug (Doxorubicin 0.55-600 μg/ml, Daunorubicin 0.07-100 μg/ml, Docetaxel 0.75-800 μg/ml, Paclitaxel 0.5-500 μg/ml, Imatinib 4.5-5000 μg/ml, Cisplatin 0.04-50 μg/ml) or medium alone was added to the wells. The plates were returned to the incubator for 48 hours following which cell titre blue assays were carried out.

Effect of Mycograb in Combination with Anti-cancer Agents Doxorubicin, Daunorubicin, Docetaxel, Paclitaxel, Imatinib, Cisplatin and Hydroxyurea on K562 Cells

The cell lines were counted. 100 μl of 2×10⁵, or 4×10⁵ cells/ml were added to 96 well flat bottomed tissue culture plates. The plates were then incubated overnight at 37° C., 5% CO₂. 100 μl of fresh medium was added to the plates and a checkerboard of Mycograb versus other drug was set out as outlined in Table 9 below (using Doxorubicin as an example) to give a total volume of 200 μl per well.

Experiments were also performed with KU-812 cells using the above methodologies, and results given below.

Results

Effect of Mycograb® on K562 Cells

With cell line K562 Mycograb on its own at 12.5 μg/ml demonstrated a 40% reduction in cell viability.

Effect of Mycograb® and Anti-cancer Agents on K562 Cells

Imatinib

The IC₅₀ was 16 μg/ml. There was some evidence of synergy between Imatinib and Mycograb® at a range of drug concentrations (see Table 10).

Doxorubicin

The IC₅₀ was 1 μg/ml. There was some evidence of synergy between Doxorubicin and Mycograb® at some drug concentrations but mostly indifference with Mycograb®.

Daunorubicin

The IC₅₀ was 0.75 μg/ml. There was some evidence of synergy between Daunorubicin and Mycograb® at low of drug concentrations (see Table 11).

Docetaxel

The IC₅₀ was 70 μg/ml. There was clear evidence of synergy between Docetaxel and Mycograb® at a range of drug concentrations (see Table 12).

Paclitaxel

The IC₅₀ was 32 μg/ml. There was clear evidence of synergy between Paclitaxel and Mycograb® at a range of drug concentrations (see Table 13).

Cisplatin

The IC₅₀ was 12.5 μg/ml. There was no evidence of synergy between Cisplatin and Mycograb®.

Hydroxyurea

The IC₅₀ was never reached with Hydroxyurea as the sole agent. However, there was some evidence of synergy between Hydroxyurea and Mycograb® at low of drug concentrations (see Table 14).

KU-812 Cell line

Effect of Mycograb® on KU-812 Cells

With cell line KU-812 Mycograb on its own at 50 μg/ml demonstrated a 40% reduction in cell viability.

Effect of Mycograb® and Anti-Cancer Agents on KU-812 Cells

Imatinib

The IC₅₀ was 0.12 μg/ml. There was some evidence of synergy between Imatinib and Mycograb® at low of drug concentrations (see Table 15).

Summary

Using a K562 cell line, there was evidence of synergy with Imatinib, Paclitaxel, and Docetaxel. There was evidence of some synergy with Daunorubicin, Doxorubicin and Hydroxyurea. Indifference was seen with Cisplatin.

Using a KU-812 cell line, there was evidence of some synergy with Imatinib.

Conclusions

The data presented here clearly demonstrates that Mycograb® antibody on it's own can decrease the viability of both Ph1-positive and Ph1-negative CML cell lines. Furthermore, there is a surprising synergism between anti-cancers agents, including Imatinib and the anti-Hsp90 antibody, in Ph1-positive CML cell lines. The data also demonstrate that there is synergism between Paclitaxel, Docetaxel, Daunorubicin, Doxorubicin, Hydroxyurea, and the anti-Hsp90 antibody, in Ph1-positive leukaemia cell lines. These results allows for the use of compositions comprising anti-cancer agents such as Imatinib, together with the anti-Hsp90 antibody (Mycograb,®) for the treatment of CML. The synergism exhibited by the combination of anti-cancer agent and Mycograb® antibody potentially allows for either lower treatment dosages, which would be hugely significant given the problematic toxicity of many of the anti-cancer agents, and in particular Imatinib, or more effective and longer treatments at the same dosages, thereby reducing unwanted side-effects.

Clinical implications of the present invention include: (i) the production of a synergistic combination of anti-cancer agents e.g. Imatinib, and anti-Hsp90 antibody in the treatment of CML should become the treatment of choice. This would possibly lead to a reduction in mortality for CML; (ii) Imatinib is toxic, and the synergy provided by the present invention means that a lower dose of Imatinib could be used while maintaining efficacy and concomitantly reducing toxicity; and (iii) the toxicity sparing effect of the anti-hsp90 antibody would allow the clinical efficacy of higher doses of Imatinib to be explored and further contribute to an improved clinical outcome.

### Experiments C

A third set of experiments (described below) detail the investigation of the effect of an anti-Hsp90 antibody having the sequence SEQ ID NO: 2 and specific for the epitope displayed by a peptide having the sequence SEQ ID NO: 1, used on its own or in combination with the anti-cancer agents 5-FU and Folinic acid and/or Oxaliplatin on the human colon adenocarcinoma cell line HT29.

The results show that the antibody gave evidence of synergy with 5-FU and Folinic acid and with Oxaliplatin. There was also evidence of synergy with the four drug combination of Mycograb/anti-Hsp90 antibody with 5-FU, Folinic acid and Oxaliplatin. Concentrations of greater then 75 μg/ml of 5-FU and 10.5 μg/ml of Oxaliplatin were found to be particularly useful.

Material and Methods

Cell Line and Culture Information

Human Caucasian colon adenocarcinoma grade 11 cell line HT29, was obtained from ECACC (ECACC number 91072201).

Cells were split using 0.25% trypsin/EDTA (Sigma) and maintained in McCoy's 5a medium containing 10% Foetal Bovine Serum, 2 mM Glutamine, 100U Penicillin, 0.1 mg Streptomycin (Sigma) at 37° C., 5% CO₂.

Other cell lines include HCT116

Experiments

Effect of Mycograb® on HT29 Cells

The cell lines were split and cells the counted. Cells were added to 12- or 96-well flat-bottomed tissue culture plates. In the case of the 12-well plates, 1 ml of 4×10⁴ cells/ml or 4×10⁵ cells/ml were added to each well plus 1 ml of complete McCoy's 5a medium. In the case of the 96-well plate, 100 μl of 4×10⁴ cells/ml or 4×10⁵ cells/ml were added followed by a further 100 μl of complete McCoy's 5a medium was added to each well. The plates were then incubated overnight at 37° C., 5% CO₂. The next day, the cells were observed under a phase contrast microscopy to ensure they had adhered to the plates and the supernatant medium removed by aspiration. Fresh complete RPMI medium containing two-fold increasing concentrations of Mycograb® (1.5-200 μg/ml) or medium alone was then added to the wells. The plate was returned to the incubator for 48 hours following which the cell titre blue assay was carried out or viable counts were completed.

Effect of Anti-Cancer Agents 5FU and Folinic Acid and Oxaliplatin on HT29 Cells

The cell lines were split and the cells counted. 100 μl of 4×10⁴ cells/ml or 4×10⁵ cells/ml were added to 96 well flat bottomed tissue culture plates a further 100 μl of complete McCoy's 5a medium was added to the plate. The plates were then incubated overnight at 37° C., 5% CO₂. The next day, the cells were observed under phase contrast microscopy to ensure they had adhered to the plates and the supernatant medium removed by aspiration. 100 μl of fresh complete RPMI medium containing increasing two fold concentrations of study drug (5-FU 4.5-2400 μg/ml plus 1 mg/ml Folinic acid or Oxaliplatin 1-500 μg/ml) or medium alone (Media+2.5% DMSO for 5-FU control) was then added to the wells. The plates were returned to the incubator for 48 hours following which cell titre blue assays were carried out.

Effect of Mycograb® in Combination with Anti-Cancer Agents 5FU and Folinic Acid or Oxaliplatin on HT29 Cells

The cell lines were split and the cells counted. 100 μl of 4×10⁴ cells/ml or 4×10⁵ cells/ml were added to 96 well flat bottomed tissue culture plates a further 100 μl of complete McCoy's 5a medium was added to the plate. The plates were then incubated overnight at 37° C., 5% CO₂. The next day, the cells were observed under phase contrast microscopy to ensure they had adhered to the plates and the supernatant medium removed by aspiration. 100 μl of fresh complete RPMI medium was added to the plates and a checkerboard of Mycograb®versus study drug ((5-FU 4.5-2400 μg/ml plus 1 mg/ml Folinic acid or Oxaliplatin 1-500 μg/ml) or medium alone (Medium+2.5% DMSO for 5-FU control)) was set out as outlined in Table 16 to give a total volume of 200 μl per well.

Effect of Mycograb® in Combination with Anti-Cancer Agents 5FU and Folinic Acid and Oxaliplatin on HT29 Cells

The cell lines were split and the cells counted. 100 μl of 4×10⁴ cells/ml or 4×10⁵ cells/ml were added to 96 well flat bottomed tissue culture plates a further 100 μl of complete McCoy's 5a medium was added to the plate. The plates were then incubated overnight at 37° C., 5% CO₂. The next day, the cells were observed under phase contrast microscopy to ensure they had adhered to the plates and the supernatant medium removed by aspiration. 100 μl of fresh complete RPMI medium was added to the plates and a checkerboard of Mycograb® versus study drug ((5-FU:Folinic acid:Oxaliplatin at a ratio of 3:1:0.42) or medium alone (Medium+2.5% DMSO for 5-FU control)) was set out as for the 5-FU checkerboard outlined in Table 16 to give a total volume of 200 μl per well.

Results

Effect of Mycograb® on HT29 Cells

With cell line HT29 Mycograb® on its own at 125 μg/ml demonstrated a 50% reduction in cell viability.

Effect of Anti-Cancer Agents 5Fu or Oxaliplatin Alone and Mycograb® in Combination with Anti-Cancer Agents 5Fu or Oxaliplatin on HT29 Cells

5-FU:

The IC₅₀ for 5-Fluorouracil was 150 μg/ml. There was clear evidence of synergy between 5-FU and Mycograb® at a range of drug concentrations see Tables 17-20.

Oxaliplatin:

The IC₅₀ for Oxaliplatin was 16 μg/ml. There was some evidence of synergy between Oxaliplatin and Mycograb® at a range of drug concentrations, Table 18.

Effect of Mycograb® in Combination with Anti-Cancer Agents 5FU and Oxaliplatin on HT29 Cells

The IC₅₀ of LV/5FU/Ox was 25/75/10.5 μg/ml. There was some evidence of synergy between 5-FU and Oxaliplatin and Mycograb® at a range of drug concentrations see Tables 22-24.

Summary

The results show that the antibody gave evidence of synergy with 5-FU and Folinic acid or Oxaliplatin and some evidence of synergy with the four drug combination of with 5-FU and Folinic acid and Oxaliplatin with concentrations of greater then 75 μg/ml 5-FU and greater than 10.5 μg/ml Oxaliplatin. There was evidence of synergy with 5-FU and Oxaliplatin, Table 25 summarises the CI values at ED₅₀, ED₇₅ and ED₉₀.

Conclusions

The data presented here demonstrates that Mycograb® antibody on its own can decrease the viability of a colon adenocarcinoma cell line. There was synergy between anti-cancer agents, including 5-Fluorouracil and Oxaliplatin and the anti-HSP 90 antibody in a colon adenocarcinoma cell line. The data also demonstrates synergy between 5-Fluorouracil and Oxaliplatin with the anti-HSP 90 antibody in a colon adenocarcinoma cell line.

TABLE 1
Checkerboard of Mycograb (MG) (μg/ml) versus Doxorubicin (DR) (μg/ml)

| | 1 | 2 | 3 | 4 | 5 | 6 | 7 | 8 | 9 | 10 | 11 | 12 |
|---|---|---|---|---|---|---|---|---|---|---|---|---|
| A | DR 150 | DR 75 | DR 37 | DR 18.5 | DR 9 | DR 4.5 | DR 2.3 | DR 1.12 | DR 0.55 | DR 0.27 | MG 50 | Media, |
| | MG 50 | MG 50 | MG 50 | MG 50 | MG 50 | MG 50 | MG 50 | MG 50 | MG 50 | MG 50 | | no cells |
| B | DR 150 | DR 75 | DR 37 | DR 18.5 | DR 9 | DR 4.5 | DR 2.3 | DR 1.12 | DR 0.55 | DR 0.27 | MG 25 | Media, |
| | MG 25 | MG 25 | MG 25 | MG 25 | MG 25 | MG 25 | MG 25 | MG 25 | MG 25 | MG 25 | | no cells |
| C | DR 150 | DR 75 | DR 37 | DR 18.5 | DR 9 | DR 4.5 | DR 2.3 | DR 1.12 | DR 0.55 | DR 0.27 | MG 12.5 | Media, |
| | MG 12.5 | MG 12.5 | MG 12.5 | MG 12.5 | MG 12.5 | MG 12.5 | MG 12.5 | MG 12.5 | MG 12.5 | MG 12.5 | | no cells |
| D | DR 150 | DR 75 | DR 37 | DR 18.5 | DR 9 | DR 4.5 | DR 2.3 | DR 1.12 | DR 0.55 | DR 0.27 | MG 6.25 | Media, |
| | MG 6.25 | MG 6.25 | MG 6.25 | MG 6.25 | MG 6.25 | MG 6.25 | MG 6.25 | MG 6.25 | MG 6.25 | MG 6.25 | | no cells |
| E | DR 150 | DR 75 | DR 37 | DR 18.5 | DR 9 | DR 4.5 | DR 2.3 | DR 1.12 | DR 0.55 | DR 0.27 | MG 3 | Media, |
| | MG 3 | MG 3 | MG 3 | MG 3 | MG 3 | MG 3 | MG 3 | MG 3 | MG 3 | MG 3 | | no cells |
| F | DR 150 | DR 75 | DR 37 | DR 18.5 | DR 9 | DR 4.5 | DR 2.3 | DR 1.12 | DR 0.55 | DR 0.27 | MG 1.5 | Media, |
| | MG 1.5 | MG 1.5 | MG 1.5 | MG 1.5 | MG 1.5 | MG 1.5 | MG 1.5 | MG 1.5 | MG 1.5 | MG 1.5 | | no cells |
| G | DR 150 | DR 75 | DR 37 | DR 18.5 | DR 9 | DR 4.5 | DR 2.3 | DR 1.12 | DR 0.55 | DR 0.27 | MG 0.75 | Media, |
| | MG 0.75 | MG 0.75 | MG 0.75 | MG 0.75 | MG 0.75 | MG 0.75 | MG 0.75 | MG 0.75 | MG 0.75 | MG 0.75 | | no cells |
| H | DR 150 | DR 75 | DR 37 | DR 18.5 | DR 9 | DR 4.5 | DR 2.3 | DR 1.12 | DR 0.55 | DR 0.27 | Media | Media, |
| | | | | | | | | | | | plus cells | no cells |

TABLE 2

| Cisplatin | Mycograb | CI |
|---|---|---|
| 12.5 | 25 | 0.012 |
| 25 | 50 | 0.024 |

TABLE 3

| Mycograb | Docetaxel | CI |
|---|---|---|
| 12.5 | 100 | 0.400 |
| 25 | 200 | 0.098 |
| 50 | 400 | 0.002 |

TABLE 4

| Mycograb | Doxorubicin | CI |
|---|---|---|
| 0.75 | 2.25 | 0.249 |
| 1.5 | 4.5 | 0.261 |
| 3 | 9 | 0.248 |
| 6 | 18 | 0.476 |
| 12.5 | 37.5 | 0.186 |
| 25 | 75 | 0.155 |
| 50 | 150 | 0.159 |

TABLE 5

| Mycograb | Daunorubicin | CI |
|---|---|---|
| 0.75 | 0.75 | 0.189 |
| 1.5 | 1.5 | 0.257 |
| 3 | 3 | 0.512 |
| 6 | 6 | 0.676 |
| 12.5 | 12.5 | 0.469 |
| 25 | 25 | 0.082 |
| 50 | 50 | 0.176 |

TABLE 6

| Doxorubicin | Mycograb | CI |
|---|---|---|
| 0.55 | 0.75 | 0.573 |
| 1.12 | 1.5 | 0.541 |
| 2.25 | 3 | 0.824 |
| 4.5 | 6 | 0.254 |
| 9 | 12 | 0.507 |
| 18.5 | 25 | 0.538 |
| 37 | 50 | 1.033 |

TABLE 7

| Mycograb | Daunorubicin | CI |
|---|---|---|
| 0.75 | 0.75 | 1.153 |
| 1.5 | 1.5 | 1.300 |
| 3 | 3 | 1.557 |
| 6 | 6 | 0.763 |
| 12.5 | 12.5 | 0.367 |
| 25 | 25 | 1.014 |

TABLE 8

| Mycograb | Herceptin | CI |
|---|---|---|
| 0.75 | 0.75 | 0.007 |
| 1.5 | 1.5 | 0.008 |
| 3 | 3 | 0.005 |
| 6 | 6 | 0.034 |
| 12.5 | 12.5 | 0.025 |
| 25 | 25 | 0.170 |

TABLE 9
Checkerboard of Mycograb (MG) (μg/ml) versus Doxorubicin (DR) (μg/ml)

| | 1 | 2 | 3 | 4 | 5 | 6 | 7 | 8 | 9 | 10 | 11 | 12 |
|---|---|---|---|---|---|---|---|---|---|---|---|---|
| A | Media, | DR 0.27 | DR 0.55 | DR 1.12 | DR 2.3 | DR 4.5 | DR 9 | DR 18.5 | DR 37 | DR 75 | DR 150 | Media, |
| | with cells | | | | | | | | | | | no cells |
| B | MG 0.75 | DR 0.27 | DR 0.55 | DR 1.12 | DR 2.3 | DR 4.5 | DR 9 | DR 18.5 | DR 37 | DR 75 | DR 150 | Media, |
| | | MG 0.75 | MG 0.75 | MG 0.75 | MG 0.75 | MG 0.75 | MG 0.75 | MG 0.75 | MG 0.75 | MG 0.75 | MG 0.75 | no cells |
| C | MG 1.5 | DR 0.27 | DR 0.55 | DR 1.12 | DR 2.3 | DR 4.5 | DR 9 | DR 18.5 | DR 37 | DR 75 | DR 150 | Media, |
| | | MG 1.5 | MG 1.5 | MG 1.5 | MG 1.5 | MG 1.5 | MG 1.5 | MG 1.5 | MG 1.5 | MG 1.5 | MG 1.5 | no cells |
| D | MG 3 | DR 0.27 | DR 0.55 | DR 1.12 | DR 2.3 | DR 4.5 | DR 9 | DR 18.5 | DR 37 | DR 75 | DR 150 | Media, |
| | | MG 3 | MG 3 | MG 3 | MG 3 | MG 3 | MG 3 | MG 3 | MG 3 | MG 3 | MG 3 | no cells |
| E | MG 6.25 | DR 0.27 | DR 0.55 | DR 1.12 | DR 2.3 | DR 4.5 | DR 9 | DR 18.5 | DR 37 | DR 75 | DR 150 | Media, |
| | | MG 6.25 | MG 6.25 | MG 6.25 | MG 6.25 | MG 6.25 | MG 6.25 | MG 6.25 | MG 6.25 | MG 6.25 | MG 6.25 | no cells |
| F | MG 12.5 | DR 0.27 | DR 0.55 | DR 1.12 | DR 2.3 | DR 4.5 | DR 9 | DR 18.5 | DR 37 | DR 75 | DR 150 | Media, |
| | | MG 12.5 | MG 12.5 | MG 12.5 | MG 12.5 | MG 12.5 | MG 12.5 | MG 12.5 | MG 12.5 | MG 12.5 | MG 12.5 | no cells |
| G | MG 25 | DR 0.27 | DR 0.55 | DR 1.12 | DR 2.3 | DR 4.5 | DR 9 | DR 18.5 | DR 37 | DR 75 | DR 150 | Media, |
| | | MG 25 | MG 25 | MG 25 | MG 25 | MG 25 | MG 25 | MG 25 | MG 25 | MG 25 | MG 25 | no cells |
| H | MG 50 | DR 0.27 | DR 0.55 | DR 1.12 | DR 2.3 | DR 4.5 | DR 9 | DR 18.5 | DR 37 | DR 75 | DR 150 | Media, |
| | | MG 50 | MG 50 | MG 50 | MG 50 | MG 50 | MG 50 | MG 50 | MG 50 | MG 50 | MG 50 | no cells |

TABLE 10

| Imatinib (μg/ml) | Mycograb (μg/ml) | CI |
|---|---|---|
| 1 | 0.75 | 0.033 |
| 2 | 1.5 | 0.069 |
| 4 | 3 | 0.114 |
| 8 | 6 | 0.218 |
| 16 | 12.5 | 0.366 |
| 32 | 25 | 0.558 |
| 64 | 50 | 0.799 |

TABLE 11

| Daunorubicin (μg/ml) | Mycograb (μg/ml) | CI |
|---|---|---|
| 0.75 | 1.5 | 0.696 |
| 1.5 | 3 | 0.872 |

TABLE 12

| Docetaxel (μg/ml) | Mycograb (μg/ml) | CI |
|---|---|---|
| 1.5 | 1.5 | 0.001 |
| 3 | 3 | 0.008 |
| 6 | 6 | 0.707 |
| 12.5 | 12.5 | 0.051 |
| 25 | 25 | 0.013 |
| 50 | 50 | 0.003 |
| 100 | 100 | 0.004 |

TABLE 13

| Paclitaxel (μg/ml) | Mycograb (μg/ml) | CI |
|---|---|---|
| 1 | 1.5 | 0.064 |
| 2 | 3 | 0.145 |
| 4 | 6 | 0.011 |
| 8 | 12.5 | 0.05 |
| 16 | 25 | 0.04 |
| 32 | 50 | 0.06 |
| 64 | 100 | 0.077 |

TABLE 14

| Hydroxyurea (μg/ml) | Mycograb (μg/ml) | CI |
|---|---|---|
| 0.3 | 0.75 | 0.798 |
| 0.6 | 0.75 | 0.694 |

TABLE 15

| Imatinib (μg/ml) | Mycograb (μg/ml) | CI |
|---|---|---|
| 1 | 0.375 | 0.131 |
| 2 | 1.5 | 0.093 |
| 4 | 3 | 0.016 |
| 8 | 6 | 0.001 |
| 16 | 12.5 | 0.002 |
| 32 | 25 | 0.327 |

TABLE 16
Checkerboard of Mycograb (RTM) (MG in μg/ml) versus 5-Fluorouracil (5FU in μg/ml )

| | 1 | 2 | 3 | 4 | 5 | 6 | 7 | 8 | 9 | 10 | 11 | 12 |
|---|---|---|---|---|---|---|---|---|---|---|---|---|
| A | Media | 5FU 4.5 | 5FU 9 | 5FU 18.5 | 5FU 37 | 5FU 75 | 5FU 150 | 5FU 300 | 5FU 600 | 5FU | 5FU | Media |
| | 2.5% | | | | | | | | | 1200 | 2400 | only, no |
| | DMSO | | | | | | | | | | | cells |
| B | 2.5% | 5FU 4.5 | 5FU 9 | 5FU 18.5 | 5FU 37 | 5FU 75 | 5FU 150 | 5FU 300 | 5FU 600 | 5FU | 5FU | Media |
| | DMSO | MG 3 | MG 3 | MG 3 | MG 3 | MG 3 | MG 3 | MG 3 | MG 3 | 1200 | 2400 | only, no |
| | MG 3 | | | | | | | | | MG 3 | MG 3 | cells |
| C | 2.5% | 5FU 4.5 | 5FU 9 | 5FU 18.5 | 5FU 37 | 5FU 75 | 5FU 150 | 5FU 300 | 5FU 600 | 5FU | 5FU | Media |
| | DMSO | MG 6 | MG 6 | MG 6 | MG 6 | MG 6 | MG 6 | MG 6 | MG 6 | 1200 | 2400 | only, no |
| | MG 6 | | | | | | | | | MG 6 | MG 6 | cells |
| D | 2.5% | 5FU 4.5 | 5FU 9 | 5FU v | 5FU 37 | 5FU 75 | 5FU 150 | 5FU 300 | 5FU 600 | 5FU | 5FU | Media |
| | DMSO | MG 12.5 | MG 12.5 | MG 12.5 | MG 12.5 | MG 12.5 | MG 12.5 | MG 12.5 | MG 12.5 | 1200 | 2400 | only, no |
| | MG 12.5 | | | | | | | | | MG 12.5 | MG 12.5 | cells |
| E | 2.5% | 5FU 4.5 | 5FU 9 | 5FU 18.5 | 5FU 37 | 5FU 75 | 5FU 150 | 5FU 300 | 5FU 600 | 5FU | 5FU | Media |
| | DMSO | MG 25 | MG 25 | MG 25 | MG 25 | MG 25 | MG 25 | MG 25 | MG 25 | 1200 | 2400 | only, no |
| | MG 25 | | | | | | | | | MG 25 | MG 25 | cells |
| F | 2.5% | 5FU 4.5 | 5FU 9 | 5FU 18.5 | 5FU 37 | 5FU 75 | 5FU 150 | 5FU 300 | 5FU 600 | 5FU | 5FU | Media |
| | DMSO | MG 50 | MG 50 | MG 50 | MG 50 | MG 50 | MG 50 | MG 50 | MG 50 | 1200 | 2400 | only, no |
| | MG 50 | | | | | | | | | MG 50 | MG 50 | cells |
| G | 2.5% | 5FU 4.5 | 5FU 9 | 5FU 18.5 | 5FU 37 | 5FU 75 | 5FU 150 | 5FU 300 | 5FU 600 | 5FU | 5FU | Media |
| | DMSO | MG 100 | MG 100 | MG 100 | MG 100 | MG 100 | MG 100 | MG 100 | MG 100 | 1200 | 2400 | only, no |
| | MG 100 | | | | | | | | | MG 100 | MG 100 | cells |
| H | 2.5% | 5FU 4.5 | 5FU 9 | 5FU 18.5 | 5FU 37 | 5FU 75 | 5FU 150 | 5FU 300 | 5FU 600 | 5FU | 5FU | Media |
| | DMSO | MG 200 | MG 200 | MG 200 | MG 200 | MG 200 | MG 200 | MG 200 | MG 200 | 1200 | 2400 | only, no |
| | MG 200 | | | | | | | | | MG 200 | MG 200 | cells |

TABLE 17
5-Fluorouracil and Mycograb at a ratio of 0.37:1

| 5-FU | Mycograb | |
|---|---|---|
| (ug/ml) | (ug/ml) | CI |
| 4.5 | 12.5 | 0.001 |
| 9 | 25 | 0.004 |
| 18.5 | 50 | 0.008 |
| 37 | 100 | 0.049 |

TABLE 18
5-Fluorouracil and Mycograb at a ratio of 0.75:1

| 5-FU | Mycograb | |
|---|---|---|
| (ug/ml) | (ug/ml) | CI |
| 4.5 | 6 | 0.184 |
| 9 | 12.5 | 0.295 |

TABLE 19
5-Fluorouracil and Mycograb at a ratio of 12:1

| 5-FU | Mycograb | |
|---|---|---|
| (ug/ml) | (ug/ml) | CI |
| 37 | 3 | 0.557 |
| 75 | 6 | 0.228 |
| 150 | 12.5 | 0.198 |
| 300 | 25 | 0.540 |
| 600 | 50 | 0.250 |
| 1200 | 100 | 0.478 |
| 2400 | 100 | 0.212 |

TABLE 20
5-Fluorouracil and Mycograb at a ratio of 50:1

| 5-FU | Mycograb | |
|---|---|---|
| (ug/ml) | (ug/ml) | CI |
| 75 | 1.5 | 0.004 |
| 150 | 3 | 0.342 |
| 300 | 6 | 0.482 |

TABLE 21
Oxaliplatin and Mycograb at a ratio of 1.25:1

| Oxaliplatin | Mycograb | |
|---|---|---|
| (ug/ml) | (ug/ml) | CI |
| 7.5 | 6 | 0.378 |
| 15.5 | 12.5 | 0.182 |
| 31 | 25 | 0.153 |
| 62.5 | 50 | 0.046 |
| 125 | 100 | 0.616 |
| 250 | 200 | 0.185 |

TABLE 22
5-Fluorouracil, Oxaliplatin and Mycograb at a ratio of 3:1:0.42

| 5-FU | Mycograb | Oxaliplatin | |
|---|---|---|---|
| (ug/ml) | (ug/ml) | (ug/ml) | CI |
| 9 | 3 | 1.3 | 0.053 |
| 18.5 | 6 | 2.6 | 0.109 |
| 37 | 12.5 | 5.25 | 0.239 |
| 75 | 25 | 10.5 | 3.120 |

TABLE 23
5-Fluorouracil, Oxaliplatin and Mycograb at a ratio of 3:2:0.42

| 5-FU | Mycograb | Oxaliplatin | |
|---|---|---|---|
| (ug/ml) | (ug/ml) | (ug/ml) | CI |
| 9 | 6 | 1.3 | 0.074 |
| 18.5 | 12.5 | 2.6 | 0.152 |
| 37 | 25 | 5.25 | 0.405 |
| 75 | 50 | 10.5 | 1.519 |

TABLE 24
5-Fluorouracil, Oxaliplatin and Mycograb at a ratio of 3:0.5:0.42

| 5-FU | Mycograb | Oxaliplatin | |
|---|---|---|---|
| (ug/ml) | (ug/ml) | (ug/ml) | CI |
| 9 | 1.5 | 1.3 | 0.043 |
| 18.5 | 3 | 2.6 | 0.089 |
| 37 | 6 | 5.25 | 0.184 |
| 75 | 12.5 | 10.5 | 2.202 |

TABLE 25

| | Combination Index | | | | |
|---|---|---|---|---|---|
| Drug | Values at | | | | |
| (ratio) | ED50 | ED75 | ED90 | Dm | r |
| 5-FU | N/A | N/A | N/A | 297.1493 | 0.95571 |
| Mycograb | N/A | N/A | N/A | 96.46543 | 0.87307 |
| Oxaliplatin | N/A | N/A | N/A | 27.77133 | 0.94124 |
| 5-FU/Mycograb/ | 0.77008 | 0.11419 | 0.02256 | 64.92005 | 0.87996 |
| Oxaliplatin (3:1:0.42) | | | | | |
| 5-FU/Mycograb/ | 0.85085 | 0.16415 | 0.03924 | 55.54798 | 0.97163 |
| Oxaliplatin | | | | | |
| (1.5:1:0.21) | | | | | |
| 5-FU/Mycograb/ | 0.62267 | 0.09916 | 0.02224 | 61.08042 | 0.9142 |
| Oxaliplatin (6:1:0.85) | | | | | |

1. The use of:
(i) an antibody or an antigen binding fragment thereof specific for at least one epitope of Hsp90; and
(ii) at least one anti-cancer agent selected from the group consisting of: Doxorubicin, Daunorubicin, Epirubicin, Herceptin, Docetaxel, and Cisplatin,
in a method of manufacture of a medicament for the treatment of cancer.

2. A combined preparation comprising:
(i) an antibody or an antigen binding fragment thereof specific for at least one epitope of Hsp90; and
(ii) at least one anti-cancer agent selected from the group consisting of: Doxorubicin, Daunorubicin, Epirubicin, Herceptin, Docetaxel, and Cisplatin,
for simultaneous, separate or sequential use in the treatment of cancer.

3. The combined preparation according to claim 2, wherein said antibody or antigen binding fragment thereof is specific for the epitope displayed by the peptide having the sequence of SEQ ID NO: 1.

4. The combined preparation according to claim 2, wherein said antibody comprises the sequence of SEQ ID NO: 2.

5. The combined preparation according to claim 2, wherein said cancer is selected from the group consisting of: fibrosarcoma, breast, prostate, melanoma, leukemia, lymphomas, colon, testicular germ cell, pancreatic, ovarian, endometrial, thyroid, and lung.

6. A method of treatment of cancer comprising administering a therapeutically effective quantity of:
(i) an antibody or an antigen binding fragment thereof specific for at least one epitope of Hsp90; and
(ii) at least one anti-cancer agent selected from the group consisting of: Doxorubicin, Daunorubicin, Epirubicin, Herceptin, Docetaxel, and Cisplatin,
to a patient in need of same.

7. The use of:
(i) an antibody or an antigen binding fragment thereof specific for at least one epitope of Hsp90;
in a method of manufacture of a medicament for the treatment of leukaemia.

8. The use of:
(i) an antibody or an antigen binding fragment thereof specific for at least one epitope of Hsp90; and
(ii) at least one anti-cancer agent selected from the group consisting of: Imatinib, Paclitaxel, Docetaxel, Daunorubicin, Doxorubicin, and Hydroxyurea,
in a method of manufacture of a medicament for the treatment of leukaemia.

9. A combined preparation comprising:
(i) an antibody or an antigen binding fragment thereof specific for at least one epitope of Hsp90; and
(ii) at least one anti-cancer agent selected from the group consisting of: Imatinib, Paclitaxel, Docetaxel, Daunorubicin, Doxorubicin, and Hydroxyurea,
for simultaneous, separate or sequential use in the treatment of leukaemia.

10. The combined preparation according to claim 9, wherein said antibody or antigen binding fragment thereof is specific for the epitope displayed by the peptide having the sequence of SEQ ID NO: 1.

11. The combined preparation according to claim 9, wherein said antibody comprises the sequence of SEQ ID NO: 2.

12. The combined preparation according to claim 9, wherein said leukaemia is selected from the group consisting of: acute myeloblastic leukaemia, acute lymphoblastic leukaemia, chronic myeloid leukaemia, and chronic lymphocytic leukaemia.

13. The combined preparation according to claim 9, wherein said at least one anti-cancer agent is Imatinib.

14. The combined preparation according to claim 9, wherein said leukaemia is chronic myeloid leukaemia or acute lymphoid leukaemia.

15. The combined preparation according to claim 14, wherein said leukaemia is characterized by cells which are Philadelphia chromosome positive, or cells which are Philadelphia chromosome negative.

16. The combined preparation according to claim 14, wherein said anti-cancer agent is Imatinib, and said leukaemia is characterized by cells which are Philadelphia chromosome positive.

17. The combined preparation according to claim 14, wherein said anti-cancer agent is Imatinib, and said leukaemia is characterized by cells which are Philadelphia chromosome negative.

18. The combined preparation according to claim 9, wherein said leukaemia is characterized by cells which are Imatinib resistant.

19. A method of treatment of leukaemia comprising administering a therapeutically effective quantity of:
(i) an antibody or an antigen binding fragment thereof specific for at least one epitope of Hsp90; and
(ii) at least one anti-cancer agent selected from the group consisting of: Imatinib, Paclitaxel, Docetaxel, Daunorubicin, Doxorubicin, and Hydroxyurea,
to a patient in need of same.

20. The method according to claim 19, wherein said leukaemia is chronic myeloid leukaemia, and said at least one anti-cancer agent is Imatinib.

21. The use of:
(i) an antibody or an antigen binding fragment thereof specific for at least one epitope of Hsp90; and
(ii) at least one anti-cancer agent selected from the group consisting of:
5-fluorouracil, oxaliplatin, irinotecan and raltitrexed,
in a method of manufacture of a medicament for the treatment of cancer.

22. A combined preparation comprising:
(i) an antibody or an antigen binding fragment thereof specific for at least one epitope of Hsp90; and
(ii) at least one anti-cancer agent selected from the group consisting of:
5-fluorouracil, oxaliplatin, irinotecan and raltitrexed, for simultaneous, separate or sequential use in the treatment of cancer.

23. The combined preparation according to claim 22, wherein said antibody or antigen binding fragment thereof is specific for the epitope displayed by the peptide having the sequence of SEQ ID NO: 1.

24. The combined preparation according to claim 22, wherein said antibody comprises the sequence of SEQ ID NO: 2.

25. The combined preparation according to claim 22 wherein said cancer is selected from the group consisting of: fibrosarcoma, adenocarcinoma, breast, prostate, melanoma, leukaemia, lymphomas, colon, colorectal, testicular germ cell, pancreatic, ovarian, endometrial, thyroid, and lung.

26. The combined preparation according to claim 25 wherein said cancer is colorectal cancer or adenocarcinoma.

27. A method of treatment of cancer comprising administering a therapeutically effective quantity of:
(i) an antibody or an antigen binding fragment thereof specific for at least one epitope of Hsp90; and
(ii) at least one anti-cancer agent selected from the group consisting of: 5-fluorouracil, oxaliplatin, irinotecan and raltitrexed,
to a patient in need of same.

28. The combined preparation according to claim 22 wherein the anti-cancer agent is 5-fluorouracil and further comprises folinic acid (leucovorin).

29. The combined preparation or according to claim 28 wherein the anti-cancer agent comprises 5-fluorouracil, folinic acid (leucovorin) and oxaliplatin.

30. The method according to claim 6 wherein said composition or combined preparation is administered orally.

31. The combined preparation according to claim 2, wherein said antibody or antigen binding fragment is labelled with a detectable label.

32. The combined preparation according to claim 2, wherein said antibody or antigen binding fragment is conjugated with an effector molecule.

33. (canceled)


SEQUENCE LISTING (2 sequences)

SEQ ID NO: 1 - protein, 9 aa, Candida sp.
LKVIRKNIV

SEQ ID NO: 2 - protein, 248 aa, Artificial Sequence
  Synthetic
HMAEVQLVESGAEVKKPGESLRISCKGSGCIISSYWISWVRQMPGKGLEWMGKIDPGDSYINYSPSFQGHVTISADKSINTAYLQWNSLKASDTAMYYCARGGRDFGDSFDYWGQGTLVTVSSGGGGSGGGGSGGGGSDVVMTQSPSFLSAFVGDRITITCRASSGISRYLAWYQQAPGKAPKLLIYAASTLQTGVPSRFSGSGSGTEFTLTINSLQPEDFATYYCQHLNSYPLTFGGGTKVDIKRAA